(19) United States
(12) Patent Application Publication
(10) Pub. No.: US 2003/0031650 A1
(43) Pub. Date: Feb. 13, 2003

(54) Drug inducible system and use thereof

(76) Inventors: Jacques Galipeau, Town of Mount Royal (CA); Diane E. Jaalouk, Montreal (CA); Nicoletta Eliopoulos, Montreal (CA); Clement Couture, Ville Saint-Laurent (CA); Sylvie Mader, Town of Mount-Royal (CA)

Correspondence Address: KLAUBER & JACKSON, 411 HACKENSACK AVENUE, HACKENSACK, NJ, 07601

(21) Appl. No.: 10/161,333

(22) Filed: Jun. 3, 2002

Related U.S. Application Data
(63) Continuation of application No. PCT/CA00/01422, filed on Nov. 30, 2000.
(60) Provisional application No. 60/168,299, filed on Dec. 1, 1999.

(51) Int. Cl. A61K 48/00; A61K 31/573; C12N 15/867

(52) U.S. Cl. 424/93.2; 514/179; 435/456

(57) ABSTRACT

The present invention relates to a drug inducible vector regulatable with a transactivator native to a host, and to a transplantable autologous tissue capable of engrafting in a recipient without requiring toxic conditioning, for transgene delivery to a recipient. Current drug inducible host-vector systems are responsible to foreign non-eukaryotic transcriptional activators which are potentially immunogenic and affect the long-term survival and function thereof. The present invention provides a drug inducible expression vector comprising a transgene operably linked to a reporter and to an inducible promoter responsive to a transcriptional activator of a host when exposed to an effective amount of a clinically acceptable drug. Such a vector may be introduced in a transplantable host derived from the recipient and capable of engrafting in the recipient without requiring toxic conditioning

### BACKGROUND OF THE INVENTION

(a) Field of the Invention

The present invention relates to drug inducible expression vectors and more particularly to a retrovector capable of being regulated with a native enkaryotic transactivator. The present invention also relates to a transplantable autologous tissue capable of engrafting without requiring toxic conditioning, for transgene delivery to a recipient.

(b) Description of Prior Art

Viral vectors remain the most efficient means of introducing a synthetic genetic material in cells. Among these, retroviral vectors, “gutless” adenoviruses and adeno-associated viruses are characterized by the absence of potentially immunogenic passenger viral proteins in transduced cells. Retrovectors derived from C-type mammalian retroviruses or lentiviruses are further characterized by their unique ability to stably integrate in chromosomal DNA, ensuring that vector DNA will be present in all daughter cells of the originally engineered tissue. For these reasons, retroviral vectors have been a favored means of introducing genetic material in cells where long-term transgene expression is sought (Miller, A. D. et al., Methods in Enzymology 217, 581-599 (1993)).

Integrating viral vectors are useful in gene therapy strategies where sustained transgene expression is required in all daughter cells that arise from the original genetically engineered tissue. This is exemplified by the use of replication-defective Moloney oncoretroviruses (MLV) for genetic engineering of hematopoietic stem cells, lymphocytes, and other transplantable progenitor cell types. Integrated murine leukemia virus (MLV) retrovectors are transcriptionally active in most cell types as a consequence of strong constitutive promoter activity arising from the retroviral 5′ long terminal repeat (5′LTR) (Miller, A. D. et al., Methods in Enzymology 217, 581-599 (1993)). Constitutive promoter activity is desirable in many gene therapy scenarios where the beneficial effect of a therapeutic transgene is dependent on its on-going expression.

Adding heterologous promoter and enhancer elements to the retroviral promoter can broaden retrovector transcriptional activity. The goal is obtaining improved constitutive, tumor specific or tissue specific transgene expression. However, these designs may be plagued by “promoter interference” which leads to unpredictable transcriptional activity in transduced target cells. This interference can be addressed by deactivating endogenous retroviral promoter elements, thereby self-inactivating the retroviral platform. Self-inactivating (SIN) retrovectors can be generated by removing LTR U3 enhancer elements from the plasmid expression retrovector, yet preserving cis-acting LTR elements necessary for efficient retrovector production, reverse transcription and DNA integration. The introduction of heterologous promoter elements to a SIN retrovector template can confer novel transcriptional properties to the integrated proviral DNA in the absence of interference from endogenous retroviral enhancers. Tissue specificity, tumor specificity and conditional expression have been described in such SIN templates.

Developed hybrid vectors, however, have reduced titers, are genetically unstable or experience reconstitution of the U3-deleted LTR at high frequencies.

Regulated expression is mandatory in therapeutic strategies where continuous transgene expression would be deleterious or toxic. Regulating the transcriptional activity of recombinant retrovectors involves restricting and redirecting retrovector expression subsequent to its chromosomal integration. Incorporation of heterologous enhancer and promoter elements renders this possible.

Inducible promoters can be turned on or off through the presence or absence of a particular compound or through a change in conditions such as temperature. Inducible host-vector systems responsive to exogenous stimulus have been described. Among these, the tetracycline responsive system has been validated in vivo. Rodents implanted with genetically engineered myoblasts given doxycycline-laced drinking water have measurable induction of transgene expression. The induction is reversible upon doxycycline withdrawal. Other drug inducible systems have since been reported, including the use of chimeric fusion proteins as transactivators responsive to FK506 and RU486.

The common denominator to these drug inducible systems is the requirement of a “foreign”, non-eukaryotic gene product that acts as a conditional transactivator with respect to the recipient. However, it is now recognized that the expression of “foreign” proteins by engineered autologous cells may initiate a specific immune response in immunocompetent mammals (Bonini, C. et al., Science 276, 1719-1724 (1997)) and sometimes with morbid consequences (Nature Medicine, Vol XX, 1999).

Long-term survival and function of engineered cells in vivo could be enhanced by minimizing the use of potentially antigenic foreign reporter and regulatory gene products. The same concern holds true for conditional inducible systems. Further, the drug used as a “switch” must have an acceptable clinical tolerance profile, i.e. its side-effects, if any, must be reasonable.

It would therefore be highly desirable to be provided with a drug inducible expression vector responsive to a transcriptional activator native to a host. Such a transactivator would not trigger an immune response in the recipient.

Transplantable, gene-modified, cultured autologous tissue can be used for regulated systemic delivery of therapeutic proteins in a recipient. Cultured primary cells can be gene-modified in vitro. Therefore, issues related to indiscriminate in vivo viral dissemination and inadvertent gene transfer to non-targeted tissue are rendered moot. Furthermore, since gene transfer is performed under strictly controlled conditions in cultured cells, the end product (gene transfer efficiency, and level of transgene expression) should be predictable and reproducible.

Autologous bone marrow hematopoietic stem cells are extensively used in the clinic following high-dose chemotherapy for cancer. These cells can also be harvested, cultured and gene-modified in vitro. These gene-modified cells can be subsequently returned to the donor without fear of graft rejection. Though appealing as a transgene delivery vehicle, toxic conditioning of the recipient with high-dose chemotherapy is required for successful engraftment of autologous hematopoietic cells. The same holds true for engraftment of gene-modified hematopoietic cells.

Transplantable tissue that could engraft without toxic “conditioning” regimens would therefore be desirable. Such tissue may include skin fibroblasts, myoblasts and endothelial cells. All of these cultured primary cells can be transduced with reasonable efficiency. However, skin fibroblasts are somewhat limited in their ability to proliferate in vitro. Therefore large amounts of primary cells would have to be harvested to obtain a sufficient output for reimplantation. Myoblasts are almost virtually absent from most adult animals, including humans, making their harvest for gene manipulation a challenging endeavor for most adult humans. Human umbilical vein endothelial cells have been used for cell therapy in rodent models of cancer. However, these cells can only be harvested from the umbilical vein. Consequently, there is no practical means of obtaining this type of autologous tissue from an adult.

WO9717451 discloses means for expression of large amounts of biologically active recombinant GAD65. It describes more particularly a DNA construct comprising operatively linked elements of a methanol-inducible transcriptional promoter, a DNA encoding GAD65, a transcriptional terminator, and a selectable marker.

WO9853063 discloses bone marrow stromal cells transduced in vitro with a retroviral vector encoding a Factor VII gene and the subsequent delivery of such transduced cells to immunodeficient SCID NOD mice.

Stripecke et al. (Blood, vol. 94, No. 10, Suppl. 1 (art 1 of 2), Nov. 15, 1999; abstract #791) addresses the need of providing means for the “on-demand” production of proteins such as cytokines and protein hormones in cell lines and bone marrow stromal cells. Bone marrow stromal cells are the preferred embodiments of transplantable host cells capable of engrafting without requiring toxic conditioning.

Marx et al. (Human Gene Therapy, May 10, 1999, 1163-1173) teaches that coexpression of a marker gene (GFP), and a second gene of interest (the L22 variant of the dihydrofolate reductase gene) may be achieved in cells to which a retroviral bicistronic vector containing both genes is delivered.

It would therefore be highly desirable to be provided with a transplatable autologous tissue capable of engrafting without requiring toxic conditioning.

It would particularly desirable to be provided with an autologous system for transgene delivery, which would be ubiquitous, or abundant and available in recipients of all age groups, harvested from the recipient with minimal morbidity and discomfort, manipulated and gene-modified with reasonable efficiency, to be transgene-modified and reintroduced in the recipient without requiring toxic conditioning.

### SUMMARY OF THE INVENTION

One aim of the present invention is to provide a drug inducible expression vector capable of responding to a transcriptional activator native to a host.

In accordance with the present invention, there is provided a drug inducible expression vector for transfection or administration to a host cell, comprising a transgene operably linked to a reporter and to an inducible promoter capable of responding to a transcriptional activator of the host cell when said host cell is exposed to an effective amount of a clinically acceptable drug.

The expression vector may consist of a viral vector, such as a C-type retrovirus or a lentivirus. The expression vector may be capable of integrating into a genome of the host cell.

The transcriptional activator may consist of a glucocorticoid receptor (GR), and the inducible promoter may comprise a glucocorticoid response element (GRE). For example, the inducible promoter may consist of a hybrid promoter with five tandem repeats of said GRE and a green fluorescent protein (GFP) reporter. The glucocorticoid response pathway meets the above-mentioned criteria.

The drug may consist of a steroid drug or an analog thereof, such as dexamethasone. Corticosteroids and their synthetic analogs are commonly used pharmacological agents in clinical medicine. They exert their cellular effect by interacting with the cytoplasmic glucocorticoid receptor (GR) in target cells. Steroid-bound GR subsequently transactivates target genes via the Glucocorticoid Response Elements (GRE). The GR is nearly ubiquitously distributed in normal tissue. Therefore, most normal cells likely have the ability to transactivate synthetic GRE-dependent transgenes when exposed to pharmacological amounts of corticosteroids (White, J. H. Advances in Pharmacology (New York) 40, 339-367 (1997)). This hypothesis has been validated by Mader et al. (Mader, S. & White, J. H. Proceedings of the National Academy of Sciences of the United States of America 90, 5603-5607 (1993)), who demonstrated that reporter plasmid constructs incorporating GREs would transactivate in HeLa cells exposed to dexamethasone. Moreover, this eliminates the need for a potentially immunogenic prokaryotic transactivator.

The transgene may encode a cytokine, a hormone, a growth factor, a clotting factor or a chimeric protein.

Another aim of the present invention is to provide a transplantable autologous tissue capable of engrafting in a patient without requiring toxic conditioning, and which is abundant and available in recipients of all age groups, harvested from the recipient with minimal morbidity and discomfort, manipulated and gene-modified with reasonable efficiency, to be transgene-modified and reintroduced in the recipient without requiring toxic conditioning.

In accordance with the present invention, there is provided a transplantable host cell for delivering a transgene to a patient, the host cell being derived from the patient and capable of engrafting therein without requiring toxic conditioning.

The host cell may consist of a primary cell such as a bone marrow stromal cell, a bone marrow hematopoietic stem cell, a skin fibroblast, a myoblast or an endothelial cell, and preferably a bone marrow stromal cell. Bone marrow stromal cells fulfill these criteria. Bone marrow stromal cells may be readily harvested from patients by a simple outpatient procedure. This procedure, routinely carried out by clinical hematologists, involves needle puncture of the iliac crest under local anesthesia and aspiration of a few milliliters of marrow content. Whole marrow aspirates are placed in culture and two populations distinguish themselves promptly: (i) “adherent” fibroblast-like cells and (ii) a mixture of “free-floating” hematopoietic cells. The fibroblast-like cells give rise to colonies also known as Colony Forming Units-Fibroblast (CFU-F). CFU-Fs—hereafter referred to as stroma—have mesenchymal progenitor cell properties. In fact, cultured stroma, under the appropriate conditions, can give rise to a variety of end-differentiated cell types of mesenchymal origin such as fibroblasts, adipocytes, chondrocytes, osteoblasts, myocytes and cardiomyocytes.

Stromal cells proliferate in vitro in the presence of standard growth media and may be passaged for weeks and expanded in number without loss of progenitor potential. Mouse stromal cells have been retrovirally engineered. Stromal cells have been engineered to secrete soluble recombinant proteins such as plasma clotting Factor IX and Factor VIII. Stromal cells have an important feature that distinguishes them from hematopoietic progenitor cells. That is their ability to be transplanted and engrafted without the need of “creating space” by toxic “conditioning” regimens such as chemotherapy or radiotherapy. Interestingly, cultured stromal cells transplanted by intraperitoneal injection into recipient animals give rise to differentiated mesenchymal progeny cells in almost all viscera. This suggests that cultured stromal cells retain progenitor properties and that these cells may engraft systemically in many organ compartments including marrow, spleen, lung, liver and even brain. It has also been recently shown that allogenic stromal cells are transplantable in humans (Horwitz, E. M. et al. Nature Medicine 5, 309-313 (1999)), which strongly predicts that autologous stroma is likely to behave in humans as it does in animal models. Engineered stromal cells may serve as an autologous cellular vehicle for regulatable production of therapeutic proteins or gene by-products in vivo.

In accordance with the present invention, there is further provided a system for delivering a transgene to a patient. The system comprised such a transplantable host cell transduced with such a drug inducible expression vector.

In accordance with the present invention, there is further provided a method for regulating expression of a transgene product to a patient in need of the transgene product. The method comprises introducing into a patient a system comprising a transplantable host cell derived from the patient, capable of engrafting in the recipient without requiring toxic conditioning and transformed with a drug inducible expression vector comprising a transgene operably linked to an inducible promoter capable of responding to a transcriptional activator of the host cell when exposed to an effective amount of the drug, and administering the effective amount of the drug to the host cell, the drug binding to the transcriptional activator of the host cell, thereby inducing the inducible promoter and activating expression of the transgene, whereby the expression is regulated.

The patient may have a mesenchymal disorder and may have received chemotherapy or radiotherapy prior to the transplantation. The system may be introduced in the bone marrow, spleen, lung, liver or brain of the patient.

In accordance with the present invention, there is further provided a method for obtaining a transplantable host cell for delivering a transgene to a patient.

For the purpose of the present invention, the following terms are defined below.

The expression “clinically acceptable drug” is intended to mean a drug with no or reasonable side-effects.

An “inducible promoter” is intended to mean a promoter which can be turned on or off through the presence or absence of a particular compound or through a change in conditions such as temperature.

### BRIEF DESCRIPTION OF THE DRAWINGS

FIG. 1 illustrates schematic representations of designed retrovectors. In A, pLTRGFP plasmid retrovector bears a full-length 3′LTR that incorporates all the wild type retroviral enhancer elements and promoter machinery. The CMV promoter element substituting for the U3 region in the 5′LTR drives the expression of the retroviral genome in stably transfected retroviral packaging cells leading to production of replication-defective retroparticles. In B, pSINGRE5 is an NheI-AscI mutant of pLTRGFP whereby the retroviral enhancer and promoter elements in the 3′ U3 region are replaced by 5 tandem repeats of a glucocorticoid response element (GRE) and a minimal adenovirus 2 major late promoter. Expression of the GFP reporter in stably transfected producer cells by the CMV promoter leads to the production of self-inactivated inactivated retroparticles. In C, the detailed DNA sequence of the hybrid LTR of pSINGRE5 is presented.

FIG. 2 illustrates a Southern blot analysis on vSINGRE5 transduced HeLa cells. In A, after transduction with vSINGRE5, the retrovector will integrate into the genomic DNA. Digest of genomic DNA with KpnI, which cuts once in each flanking LTR and also upstream of the GFP reporter, and subsequent probing of the Southern blot with sequences complementary to the 5′untranslated region (Probe A) and the GFP reporter (Probe B) will allow detection of integrated proviral sequences of predicted 1051 bp and 1177 bp fragment sizes respectively. In B, Southern blot analysis of vSINGRE5 transduced (+) and untransduced (−) HeLa cells with probe A (left panel) and probe B (right panel). Molecular weights are indicated.

FIG. 3 illustrates the self-inactivation of vSINGRE5 retrovector in HeLa cells. Flow cytometry analysis of mean GFP expression in transduced HeLa cells relative to untransduced cells, vSINGRE5-HeLa show an attenuated phenotype with a 39 fold (p<0.005, n=3) reduction in mean GFP fluorescence as compared to HeLa transduced with vLTRGFP, the wild-type retrovirus.

FIG. 4 illustrates the dexamethasone inducible expression of SINGRE5 retrovector design in HeLa cells. Fluorescence microscopy and flow cytometry analysis of mean GFP expression in HeLa cells transduced with vSINGRE5 relative to HeLa null cells, induction of GFP reporter expression was maximal at 72 hrs post exposure to 250 ηM of dexamethasone.

FIG. 5 illustrates the dexamethasone, long-term, regulated expression of SINGRE5 retrovector design in HeLa cells. Following dexamethasone administration for ˜48 hrs, HeLa-vSINGRE5 exhibits a dose-dependent increase in mean GFP expression with a peak 9.1±0.8 (p<0.005) fold induction at the 250 ηM dose (panel A). The system is reversible with return of GFP reporter expression to baseline level 7 days after dexamethasone withdrawal (panel B). This glucocorticoid sensitive expression system can be serially switched “on and off” over a period of weeks (panels C to E). *Difference with control with a significance of p<0.01 (Student T test, average of three experiments).

FIG. 6 illustrates a Northern blot analysis on vSINGRE5-transduced HeLa cells. In A, schematic representation of the full-length retrovector transcript with an expected size of 2.3 kb. DNA sequence complementary to the GFP reporter cDNA was used as a probe for retroviral transcript. In B, a Northern blot analysis was done on total RNA extracted from HeLa cells transduced with vSINGRE5 at various time points of 250 ηM dexamethasone administration and withdrawal. Probing for β-actin mRNA is used as an internal control for sample loading. 28S and 18S ribosomal RNA subunits are used as molecular weight markers.

FIG. 7 illustrates a flow cytometry analysis for GFP expression by vLTRGFP-transduced bone marrow stromal cells exposed to dexamethasone. In A, non-transduced rat marrow stroma. In B, vLTRGFP transduced stroma without and in C, with exposure to 250 ηM dexamethasone. Mean GFP fluorescence (units) gating all “positive” events is indicated in top right of panels.

FIG. 8 illustrates a flow cytometry analysis for GFP expression by vSINGRE5-transduced bone marrow stromal cells exposed to dexamethasone. Left sided panels show flow cytometric analysis for green fluorescence and percent GFP positive cells is shown in panel. All samples were gated identically. Right-sided panels are fluorescence microscopy caption of test samples. Photomicrograph exposure time and settings were identical for all samples. In A and D, non-transduced rat marrow stroma. In B and E, vSINGRE5-transduced stroma in C and F, vSINGRE5-transduced stroma following 6 days in 250 ηM dexamethasone. The experiment was repeated three times with similar results. The induction of GFP expression in vSINGRE5 stroma is reversible, with fluorescence returning to baseline levels 7 days following dexamethasone removal from media.

FIG. 9 illustrates dexamethasone regulated erythropoietin secretion by bone marrow stromal cells following retroviral gene transfer.

### DETAILED DESCRIPTION OF THE INVENTION

The hypothesis that self-inactivating retrovectors incorporating GREs may serve as a platform for a conditional, corticosteroid-inducible expression system was tested. The biochemical properties of a novel dexamethasone responsive retrovector is herein disclosed and its potential use in transplantable primary tissue, namely bone marrow stroma, is demonstrated.

Transplantable bone marrow stromal cells may be utilized for cell therapy of disorders including mesenchymal disorders. Furthermore, they may also be genetically engineered to express synthetic transgenes and subsequently serve as a platform for systemic delivery of therapeutic proteins in vivo.

The inherent responsiveness of most cells to corticosteroid hormones is herein exploited as a novel means of controlling synthetic transgene expression. This strategy advantageously eliminates the need for potentially immunogenic prokaryotic or chimeric transactivators. Further, synthetic corticosteroids are pharmaceutical agents that can be readily used as transgene activators in vivo.

In a first embodiment, a self-inactivating retroviral vector incorporating a hybrid promoter with 5 tandem glucocorticoid response elements (GREs) and a Green Fluorescent Protein (GFP) reporter was constructed. Vesicular Stomatitis virus G protein pseudotyped retroparticles were synthesized and utilized to transduce cells. Reporter expression was very low in retrovector engineered HeLa cells unless exposed to dexamethasone. Transcription induction was dose-dependent and reversible, with return of retroviral RNA to basal levels promptly after dexamethasone withdrawal. GFP expression may be serially turned on and off repeatedly over a period of weeks. It is also shown that primary rat bone marrow stromal cells may be efficiently engineered with the GRE-containing retrovector of the present invention, that basal reporter expression is low, and that GFP expression is dexamethasone-inducible and reversible. In sum, this novel strategy allows dexamethasone-induced, “on-demand” transgene expression from transplantable genetically-engineered autologous cells, namely bone marrow stroma.

Normal primary cells and tissue can be genetically engineered to express synthetic transgenes and may serve as cellular platforms for systemic delivery of therapeutic gene products, such as cytokines, protein hormones, growth factors, clotting factors or chimeric proteins of clinical use. However, high-level constitutive expression of many of these products will cause significant side-effects due to a constant supra-physiological activity. “On-demand” transgene expression serves as a powerful remedy to this challenge. An ideal “inducible” system is one where engineered cells and tissue bear endogenous transactivator proteins responsive to a clinically acceptable exogenous pharmacological stimulus. The endogenous glucocorticoid response pathway existing in most cells and tissue meets these criteria (White, J. H. Advances in Pharmacology (New York) 40, 339-367 (1997)). Normal tissues that express GR may upregulate expression of an engineered GRE-dependent transgene following pharmacological doses of clinically used synthetic corticosteroids. A challenge resided in genetic engineering of target cells with a vector that would stably incorporate a transgene under control of a synthetic glucocorticoid-responsive promoter.

The objective was to generate a high-titer, genetically stable, self-inactivating (SIN) retrovector that is transcriptionally silent unless induced by a synthetic corticosteroid.

Self-Inactivating Retrovector Design and Synthesis

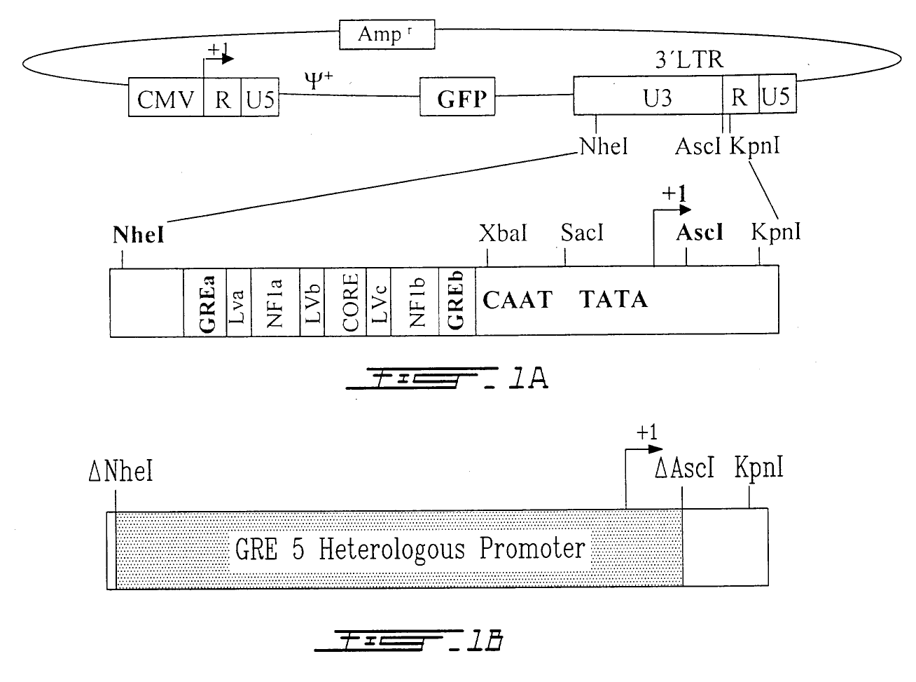

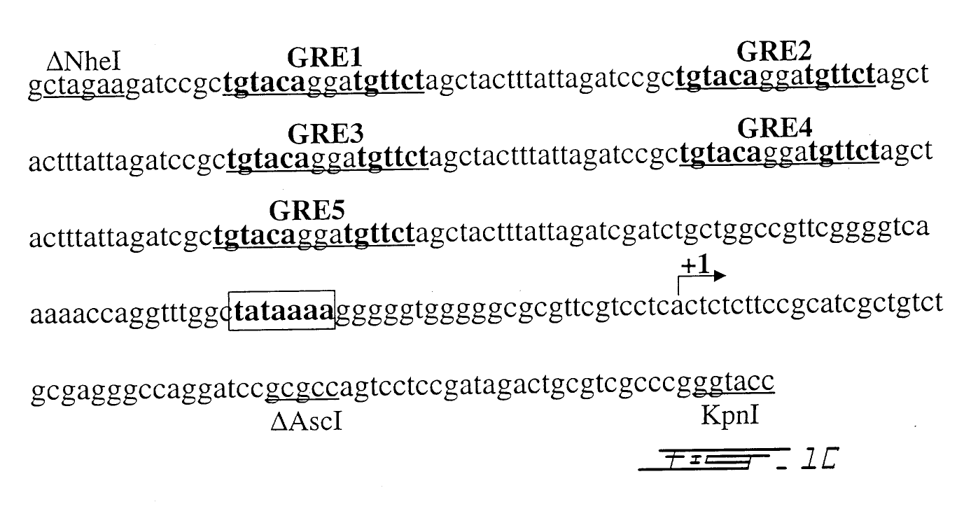

To generate a corticosteroid-responsive retrovector, a two-tiered strategy was adopted where retroviral enhancers were removed to generate a SIN template and substituted with a synthetic cluster of 5 tandem GREs. The plasmid configuration of the parental, full-length LTR retrovector construct (pLTRGFP) is depicted in FIG. 1A. Importantly, the retroparticle packaged viral RNA does not include the plasmid CMV promoter.

As shown in FIG. 1, the 3′LTR of pLTRGFP was reconfigured to render it dexamethasone-responsive by substituting all endogenous retroviral enhancers with a synthetic glucocorticoid-responsive promoter construct. The hypothesis was that basal transcriptional activity would be low or absent unless exposed to pharmacological doses of exogenous long-acting synthetic corticosteroids such as dexamethasone. A series of U3 deletion mutants was generated and the residual basal GFP reporter gene activity was measured. Among these, a NheI-XbaI SIN vector was generated that lacked retroviral enhancers, yet preserved the endogenous retroviral CAAT and TATAA boxes. It was found that the residual basal transcriptional activity was high. This entity was not further characterized. A second NheI-SacI deletion mutant removing all except the endogenous retroviral TATAA box had very little residual basal transcriptional activity and acquired dexamethasone responsiveness when added the GRE5 promoter. However, Southern blot analysis of transduced cells revealed that the proviral integrants were twice as large (˜6 kb) than the expected size (3 kb), consistent with a double genome integrant. Substituting the endogenous U3 sequences with a heterologous promoter may have destabilised the process of integration, possibly during reverse transcription.

A third deletion mutant was synthesized by deletion of a 341 bp NheI-AscI fragment spanning most of the LTR U3 region. This deletion, equivalent to removal of nt 7582 to nt 8002 of the wild-type MLV sequence (Genebank accession No. AF033811) removes all U3 enhancers, CAAT and TATA boxes, and the retroviral transcription start site. A 321 bp synthetic promoter construct incorporating 5 tandem GREs placed upstream of the adenovirus 2 major late promoter TATA box/initiation site was cloned into the U3 breach (FIG. 1B) and sequenced (FIG. 1C).

The vSINGRE5 vector generated by NheI-AscI LTR deletion differed from the previous deletion mutants by the complete absence of all endogenous retroviral promoter elements including CAAT, TATAA and transcription start. These were replaced with a synthetic GRE5 fused to a minimal Adenovirus 2 major late promoter bearing its own CAAT, TATAA and transcription start site. The pSIN(ΔNheI-AscI)GRE5 plasmid configuration allows generation of high titer virus due to the CMV/R/U5 promoter configuration in the plasmid construct as described by others. Further, the substitution of the 5′U3 retroviral sequences by the CMV promoter in the plasmid construct prevents “rescue” of the mutated 3′ LTR by homologous recombination at the DNA level.

The resulting pSIN(ΔNheI-AscI)^{GRE5}GFP construct (pSINGRE5) was used to generate stable polyclonal retroviral producer cell lines. These vSINGRE5 retroviral producer cells had a titer of ˜1×10⁵ infectious particles/ml. The retroviral supernatant was concentrated 100 fold by ultracentrifugation to a titer of ˜1×10⁷ infectious particles/ml, and utilized to transduce target cells.

Transfection of 293GPG retroviral packaging cells with pSINGRE5 plasmid led to production of VSVG-pseudotyped retroparticles at a titer of ˜1×10⁵ infectious particles/ml that were further increased 100 fold by ultracentrifugation. This allowed to deliver virus at a high MOI to cultured cells and to capitalize on the broad cross-species tropism of VSVG-pseudotyped retrovectors (Galipeau, J. et al., Cancer Research 59, 2384-2394 (1999)).

SINGRES Vector Transfer and Expression in HeLa Cells

Recombinant retroviral vectors may be susceptible to rearrangements and deletions prior to their final integration as a DNA proviral genome. Therefore, the conformation of integrated proviral vSINGRE5 DNA in transduced target cells was characterized by Southern blot analysis. Genomic DNA was extracted from transduced HeLa cells, digested with KpnI and probed with [P³²]-labeled DNA sequences complementary to the 5′untranslated proviral sequence (probe A) or to the GFP reporter cDNA (probe B).

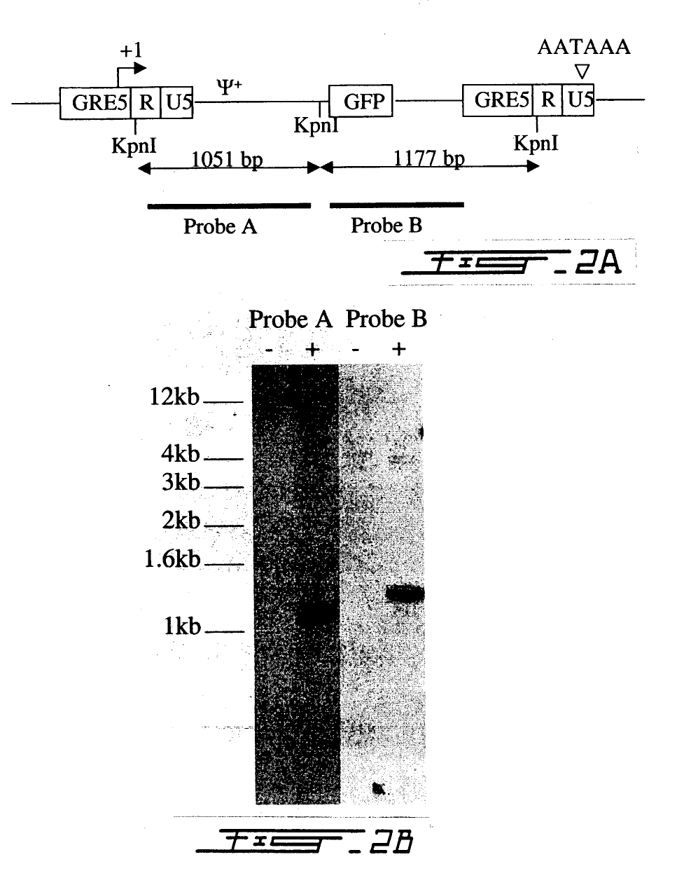

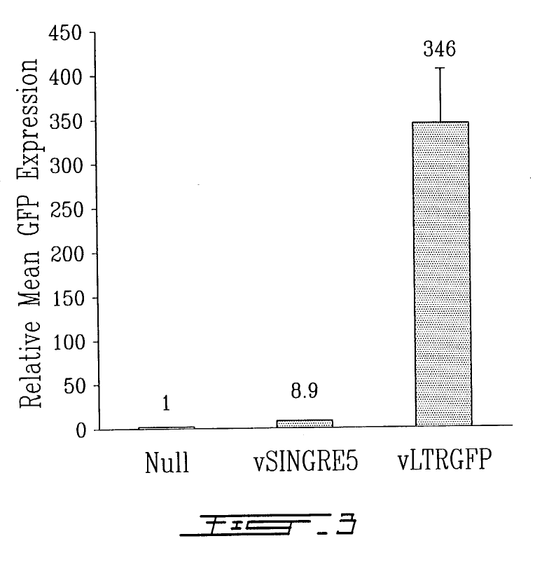

As shown in FIG. 2, DNA bands consistent with the predicted 1051 bp and 1177 bp sized fragments expected from KpnI digest of integrated unrearranged vSINGRE5 proviral DNA were detected. As shown in FIG. 3, HeLa cells transduced with vSINGRE5 integrate an unrearranged proviral genome. Interestingly, the deletion of an extra 30 bp from the ΔNheI-SacI retrovector template (removing the retroviral TATA and transcription start site shown in FIG. 1) led to stabilization of viral integration as observed with vSINGRE5 retrovector. This observation strongly suggests that the proviral instability observed with the ΔNheI-SacI SIN vector template was not from loss of essential cis-acting viral elements but rather from undefined genetic brittleness imposed by vector design.

The transcriptional activity of GFP reporter constructs can be monitored by fluorescent microscopy and flow cytometry. To determine whether the vSINGRE5 configuration led to a “self-inactivation” phenotype, the level of GFP reporter expression of vSINGRE5 was compared to that of a GFP retrovector bearing an intact LTR (vLTRGFP).

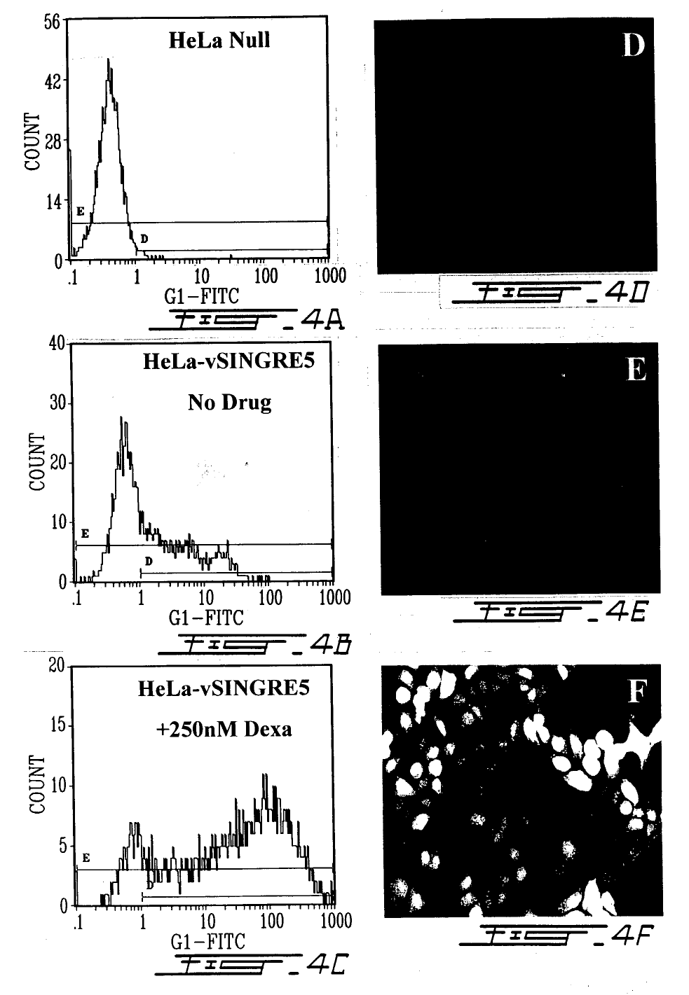

HeLa cells were transduced with vSINGRE5 or vLTRGFP at an MOI of ˜15 and GFP reporter expression was quantified by flow cytometry. Reporter expression was reduced 39 fold (p<0.005) in vSINGRE5 when compared with the vLTRGFP full-length LTR parental expression vector (FIG. 3). The vSINGRE5 HeLa cells expressed detectable but low GFP when compared with untransduced control cells (FIG. 4), and this low basal GFP expression persists despite the use of steroid-depleted media.

As shown in FIG. 3, HeLa cells transduced with vSINGRE5 express 39 times less GFP protein than equivalent unmutated vLTRGFP retrovector. This reduced basal transcriptional activity is consistent with removal of all endogenous constitutive retroviral enhancers in vSINGRE5. It is noted that measurable basal transcription activity persists despite charcoal treatment of FBS in growth media that removes all corticosteroid. As shown in FIG. 1C, the synthetic GRE5 promoter element introduced does not contain any dynamic suppressors of transcription. Therefore low level tanscriptional activity may be initiated from the minimal adenovirus 2 major late promoter, akin to what was observed in the ΔNheI-XbaI SIN template where all retroviral enhancers were removed, yet the endogenous retroviral CAAT and TATA boxes were left intact. Residual low-level transcriptional activity has also been observed with other “suppressed” regulated systems, especially when examining large polyclonal populations. It has been suggested that integrating viruses can be affected by local genomic cis-acting DNA elements such as enhancers and promoters which may directly interact with retrovector CAAT and TATAA elements. This may explain why subsets of transduced cells, all of which have different integration sites, have more basal transcriptional activity than others. Recently described “insulator” elements may minimize this phenomenon.

Synthetic Steroids and Transgene Expression

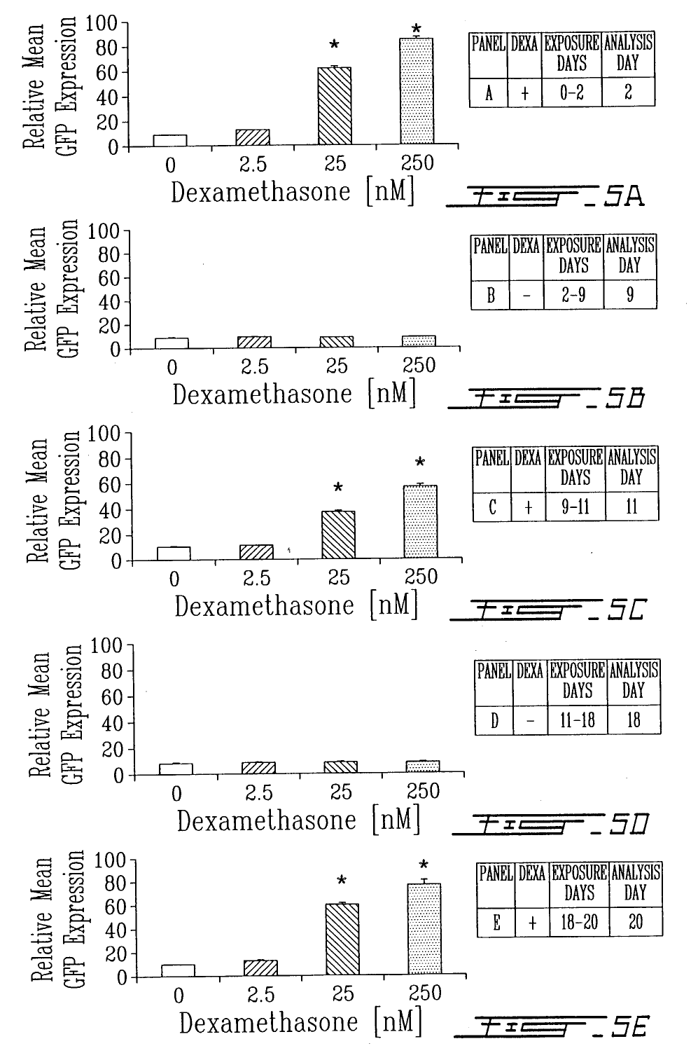

HeLa cells constitutively express the glucocorticoid receptor (GR) and co-activators that are necessary for GRE-dependent transcriptional activation after steroid exposure (Mader, S. & White, J. H. Proceedings of the National Academy of Sciences of the United States of America 90, 5603-5607 (1993)). As the vSINGRE5 retrovector construct contains 5 tandem GREs as part of its promoter makeup, it was determined whether this promoter configuration could lead to steroid-inducible transgene expression. A polyclonal population of vSINGRE5-transduced HeLa cells was exposed to 250 ηM dexamethasone for 72 hours, and GFP expression was measured by flow cytometry and documented by fluorescence microscopy (FIG. 4). GFP expression was detectable at 24 hrs and maximal at 72 hrs after drug exposure. Green fluorescence intensity, relative to untransduced HeLa cells, was also quantified by flow cytometry and utilized as a measure of GFP reporter expression level. Dose-dependent GFP induction by dexamethasone was seen with an average 1.4±0.1 (p=0.01), 6.6±0.6 (p=0.006), and 9.1±0.8 (p=0.005) fold induction in mean GFP fluorescence following 72 hrs exposure to 2.5, 25 and 250 ηM dexamethasone, respectively (FIG. 5).

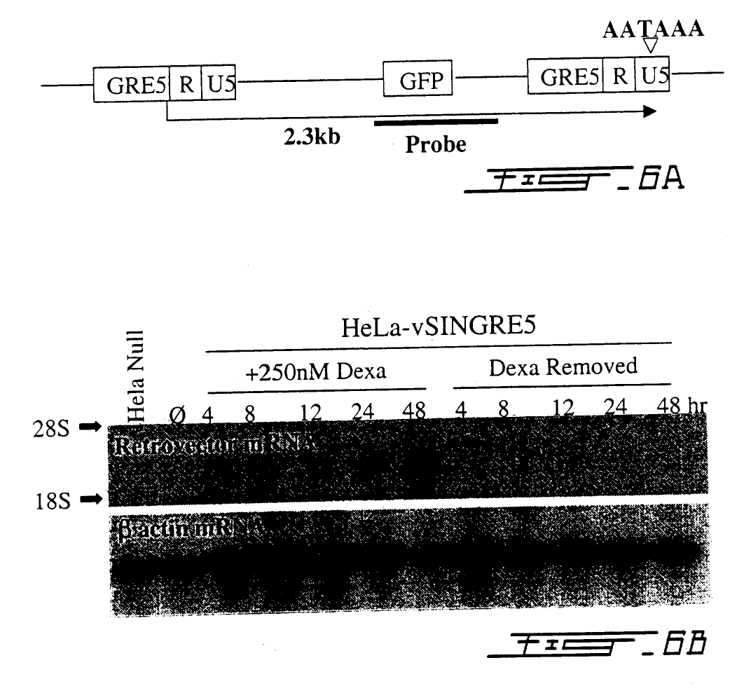

FIG. 4 shows that vSINGRE5-transduced HeLa cells can express the GFP reporter protein in a dexamethasone-responsive manner. As shown in FIG. 5, induction is dose-dependent, with low yet detectable 1.4 fold induction with 2.5 ηM dexamethasone and peak 9.1 fold induction with 250 ηM dexamethasone. Induction occurs at RNA transcription level and it is reversible (FIG. 6). Repeat inductions are feasible over weeks (FIG. 5). Lastly, the majority of unstimulated cells cultured over 40 passages retain the ability to be dexamethasone-induced, demonstrating that promoter silencing is not a significant property of this system.

Decrease of mean GFP fluorescence to basal levels was noted one week after drug removal. At least three induction/shut off cycles over a period of 1 month could be observed with comparable induction profiles (FIG. 5). A vSINGRE5-HeLa polyclonal population was maintained in continuous culture in dexamethasone-free media for more than 5 months and over 40 passages. Approximately 60% of these cells maintained their steroid responsiveness after this time.

To determine proviral RNA transcriptional kinetics, a Northern blot analysis of vSINGRE5-HeLa cells was performed after dexamethasone exposure. In dexamethasone-free media, retroviral mRNA levels in vSINGRE5-HeLa cells was below the detection threshold of our assay. Following addition of 250 ηM dexamethasone, full-length retrovector-derived MRNA was detectable at 4 hrs and peaked at 48 hrs (FIG. 6). Full-length retrovector mRNA levels decreased by ˜70% within 4 hours of dexamethasone withdrawal and returned to baseline levels within 12-24 hours.

Bone marrow stromal cells (MSCs) have recently attracted significant attention since it has been recognized that these cells have progenitor potential useful for cell therapy of mesenchymal disorders (Horwitz, E. M. et al.Nature Medicine 5, 309-313 (1999)). MSCs are appealing as vehicles for beneficial gene products as they can easily be isolated from bone marrow aspirates, expanded in vitro, transduced with viral vectors, and maintained in vivo. Therefore, autologous MSCs may serve as a cellular vehicle for in vivo delivery of therapeutic proteins (Gerson, S. L. Nature Medicine 5, 262-264 (1999)). Their engineering with a conditional, dexamethasone inducible, transgene system allows “on-demand” production of therapeutic proteins.

Transgene Induction by Dexamethasone in Engineered Bone Marrow Stromal Cells

The utility of a retroviral corticosteroid induction system is dictated by the inherent dexamethasone responsiveness of transplantable primary tissue such as bone marrow stroma. Therefore, whether gene-modified primary rat bone marrow stromal cells as a bulk population could be transcriptionally activated by exogenous dexamethasone was determined.

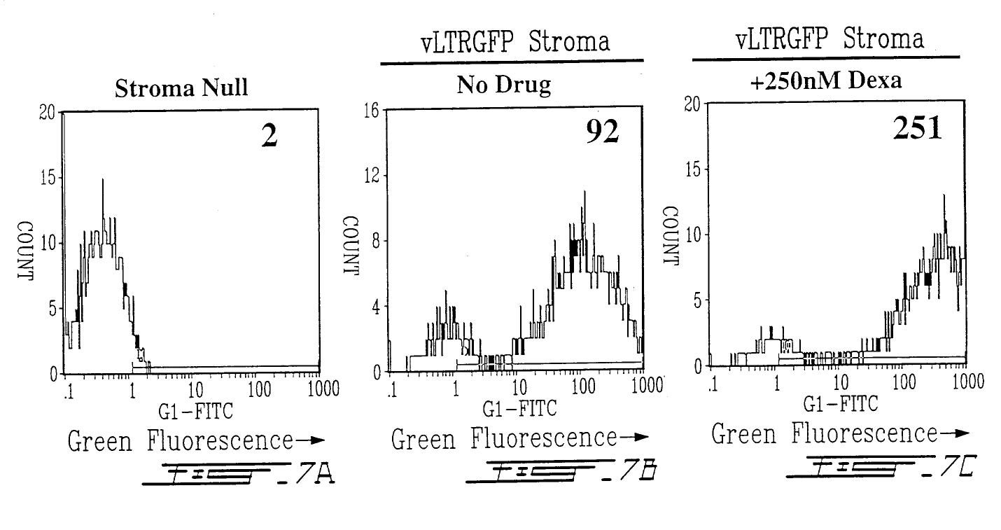

Cultured rat bone marrow stroma were transduced with high titer vLTRGFP at a MOI of 50 for two consecutive days. Gene transfer efficiency of>85% was achieved. The vLTRGFP retroviral construct, like all Moloney-based retroviruses, bears two endogenous GREs as part of its cluster of U3 enhancers (FIG. 1A). Cells transduced with a Moloney-based retrovector may therefore increase promoter activity in the presence of steroids if all necessary transactivating components, such as GR and co-activators, are present. As shown in FIG. 7, there was a 2.5 fold increase in average GFP expression in the vLTRGFP transduced stromal population following dexamethasone stimulation.

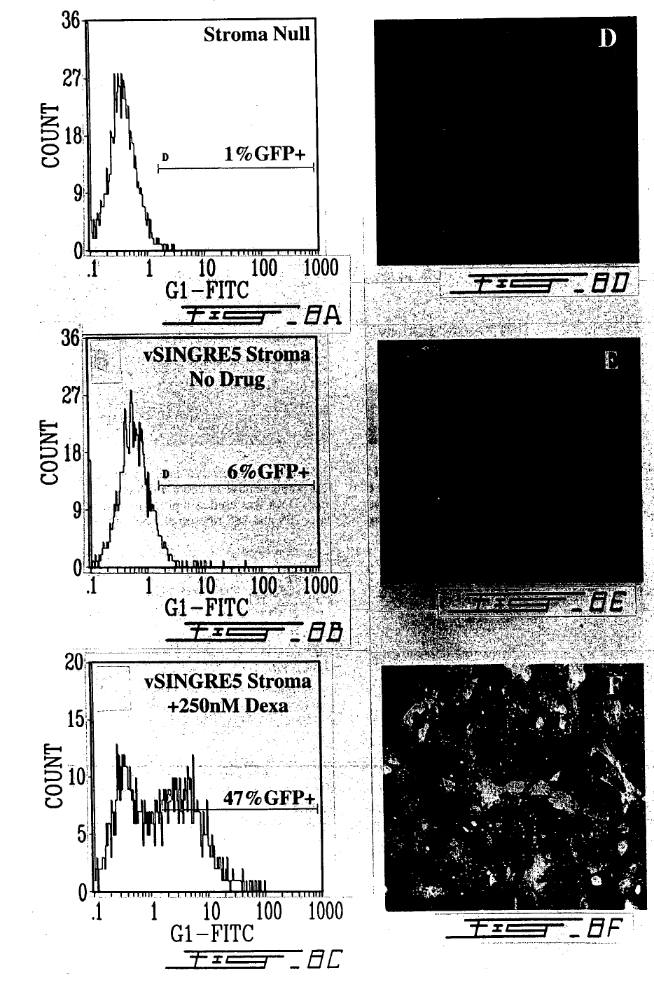

These results are consistent with the hypothesis that bulk cultured stromal cells can transactivate genes in a corticosteroid-responsive manner. This biochemical feature being a necessary premise for dexamethasone induction of vSINGRE5 expression, we determined if vSINGRE5-transduced stromal cells would be dexamethasone responsive as observed in the previously described HeLa cells. Rat marrow stroma was transduced with one application of vSINGRE5 at a final MOI of 12. Basal GFP expression was low and following exposure to 250 ηM dexamethasone for 6 days, 55% of vSINGRE5 transduced stromal cells expressed GFP (FIG. 8). The induction was reversible upon withdrawal of dexamethasone from culture medium and cells could be re-induced repeatedly over time.

FIG. 7 shows that rat marrow stroma can be readily transduced with VSVG-pseudotyped retrovectors. Gene transfer efficiency approaching 100% can be achieved when applying vLTRGFP retrovector twice at a MOI of 50. “Wild-type” Moloney retrovectors contain GREs as part of their promoter makeup (FIG. 1A) and their transcriptional activity can be increased in the presence of corticosteroids. This allows to determine if cultured stromal cells had the intrinsic capability to transactivate a GRE-dependent transgene. As seen in FIG. 7 (panels B and C), the mean GFP fluorescence of the whole vLTRGFP transduced stromal population increased 2.5 fold following dexamethasone exposure. This observation lead to conclude that, as a whole, a mixed population of cultured rat stromal cells bear the necessary transactivating machinery for corticosteroid responsiveness. A similar set of experiments was performed with vSINGRE5-transduced rat stroma. Basal GFP expression in the absence of dexamethasone was very low (FIG. 8). A substantial subset (50% of all cells), readily expressed GFP following 6 days of 250 ηM dexamethasone stimulation (FIG. 8, panels E and F). These data strongly support the idea that autologous MSCs may be used for dexamethasone-dependent transgene expression assuming that the in vivo pharmacokinetics of dexamethasone is propitious. Higher gene transfer efficiency (i.e. greater than the observed 50%) is likely to be observed in stromal cells transduced repeatedly with a high MOI of vSINGRE5 (as was achieved with vLTRGFP).

Significant MSC transgene induction in the presence of 250 ηM dexamethasone in vitro was observed. This drug concentration can be readily achieved in plasma of humans administered dexamethasone orally or intravenously. However, a theoretical concern arises from the potential transactivation of engineered stroma from endogenous corticosteroids such as cortisol. Dexamethasone differs substantially from endogenous plasma corticosteroids by its potency (25 fold higher than that of cortisol) and its prolonged biological half-life (36-72 hours versus 8-12 hours for cortisol). Therefore, physiological circadian fluctuations of plasma cortisol levels (peak 400 ηM in morning and steady state of 100 ηM, equivalent to 16 and 4 ηM of dexamethasone, respectively) would be either too transient in duration or too low in concentration to efficiently and durably transactivate vSINGRE5 engineered bone marrow stroma. Hence, genetically engineered stroma should be transcriptionally quiescent in vivo unless exposed to pharmacological doses of dexamethasone.

Retroviral vectors as gene delivery systems provide the advantage of stable transgene expression through their ability to integrate into the cellular genome, thereby ensuring that gene-modified cells and their progeny will secrete the therapeutic protein. The potential of MSCs as vehicles for the in vivo secretion of therapeutic proteins extends to all diseases where clinical improvement is conceivable via the delivery of a plasma soluble gene product or by-product. Lentiviral vector LTRs can also be self-inactivated. Lentiviral SIN vectors can also be engineered as the C-type retrovirus herein used. Dexamethasone-inducible lentiviral constructs could then be utilized to genetically engineer amitotic normal or diseased tissue in vivo including muscle, liver and brain.

In conclusion, a highly efficient and novel means of obtaining regulatable transgene expression in genetically engineered cells is provided. The corticosteroid-responsive pathway allows to exploit endogenous cellular transactivating machinery to turn on a novel steroid-responsive retroviral vector. This development significantly advances the field of inducible transgene expression since it does not depend on foreign chimeric or prokaryotic transactivators which may be immunogenic and hence a cause of graft rejection. Further, the molecular switch consists of a commonly used pharmaceutical agent (dexamethasone) with a very well characterized safety profile for use in vivo. This system can be used as is, for efficient genetic engineering of transplantable primary marrow stromal cells. Their engineering with a dexamethasone transgene could markedly enhance their penultimate in vivo therapeutic utility by allowing intermittent production of therapeutic transgenes such as growth factors, hormone, cytokines and other gene by-products. For example, rodents may be transplanted with autologous tissue engineered to secrete a therapeutic protein in a dexamethasone-responsive manner.

The present invention will be more readily understood by referring to the following examples which are given to illustrate the invention rather than to limit its scope.

### EXAMPLE I

### Cell Lines and Plasmids

pJ6ΩBleo plasmid and 293GPG retroviral packaging cell line (Ory, D. S. et al. Proceedings of the National Academy of Sciences of the United States of America 93, 11400-11406 (1996)) are from Richard. C. Mulligan, Children's Hospital, Boston, Mass. 293GPG cells are maintained in 293GPG media DMEM (Gibco-BRL, Gaithesburg, Md.), 10% heat-inactivated FBS (Gibco-BRL) supplemented with 0.3 mg/ml G418 (Mediatech, Herndon, Va.) and 2 μg/ml puromycin (Sigma, Oakville, ONT), 1 μg/ml tetracycline (Fisher Scientific, Nepean, ONT) and 50 units/ml of Pen-Strep). pAP2 plasmid (Galipeau, J. et al., Cancer Research 59, 2384-2394 (1999))and pGRE5 (Mader, S. & White, J. H. Proceedings of the National Academy of Sciences of the United States of America 90, 5603-5607 (1993)) have been previously described. HeLa and A549 cells, both human tumor cell lines, were obtained from ATCC, and were maintained in DMEM supplemented with 10% FBS and pen/strep.

### EXAMPLE II

### Retrovector Design and Synthesis

A plasmid encoding for a bicistronic, murine retrovector which incorporates a multiple cloning site—allowing insertion of cDNA of interest—linked to the Enhanced Green Fluorescence (GFP) Reporter (pAP2) (Galipeau, J. et al., Cancer Research 59, 2384-2394 (1999)) was previously described. A derivative of pAP2 was generated where the internal ribosomal entry site (IRES) was removed by classic cloning techniques. The resulting plasmid construct, pLTRGFP (FIG. 1A) contains the cDNA for the reporter enhanced GFP and a full-length LTR whose U3 region is derived from MSCV (Hawley, R. G. et al. Gene Therapy 1, 136-138 (1994)) and whose R/U5 regions are derived from pCMMPLZ, a MFG derivative. The synthesis of pSINGRE5 was as follows. The 321-bp insert encoding the cDNA for 5 glucocorticoid response elements (GRE5) and a minimal adenovirus 2 major late promoter promoter was excised by BamHI/Klenow and XbaI digest of pGRE5 (Mader, S. & White, J. H. Proceedings of the National Academy of Sciences of the United States of America 90, 5603-5607 (1993)). This insert was ligated with AscI/Klenow and NheI digest of pLTRGFP to generate pSINGRE5 plasmid (FIG. 1B). The retroviral genome for pLTRGFP and pSINGRE5 in stably transfected cells incorporates the CMV promoter element. Transduction of target cells with the derived retroviral particles (VLTRGFP or vSINGRE5) leads to the stable integration of LTR flanked proviral genome (FIG. 2). Nucleotide sequence of full-length and hybrid LTR (FIG. 1C) were confirmed by DNA sequencing (GenAlyTic Inc., University of Guelph, Ont).

### EXAMPLE III

### Production of VSVG-Pseudotyped Retroviral Particles and Virus Concentration

Recombinant VSVG-pseudotyped retroparticles were generated by stable transfection of the 293GPG packaging cell line (Ory, D. S. et al. Proceedings of the National Academy of Sciences of the United States of America 93, 11400-11406 (1996)) as previously described (Galipeau, J. et al., Cancer Research 59, 2384-2394 (1999)). In brief, stable producer cells were generated by co-transfection of 5 μg FspI linearized retrovector plasmid and pJ6ΩBleo plasmid at a 10:1 ratio. Transfected packaging cells were subsequently selected in 293GPG media supplemented with 100 μg/ml Zeocin (Invitrogen, San Diego, Calif.). Resulting stable polyclonal producer populations were utilized to generate high titer virus. All viral supernatants were filtered with 0.45 micron syringe mounted filters (Gelman Sciences, Ann Arbour, Mich.) and stored at −20° C. Concentration of VSVG retroparticles was performed as previously described (Ory, D. S. et al. Proceedings of the National Academy of Sciences of the United States of America 93, 11400-11406 (1996); Galipeau, J. et al., Cancer Research 59, 2384-2394 (1999)). In brief, previously harvested supernatant was thawed and 10 ml aliquots were centrifuged at 25,000 rpm in a SW41T1 rotor (Beckman Instruments Inc.) at 4° C. for 90 minutes. Viral pellets were resuspended overnight in 100 μl serum-free RPMI (Gibco-BRL) at 4° C. Concentrated virus was pooled, aliquoted and stored at −80° C. Viral preparations were devoid of replication competent retrovirus (RCR) by EGFP marker rescue assay utilizing conditioned supernatant collected from transduced A549 cells.

### EXAMPLE IV

### Titration of Retrovector

Target A549 cells were plated at 4×10⁴ cells per well in a 6 well tissue culture dish. The next day, cells from a test well were trypsinized and enumerated to determine baseline cell count at moment of virus exposure. Virus was serially diluted (range 100 to 0.001 μL) in a final volume of 1 ml of DMEM/10% FBS and applied to adherent cells. Flow cytometric analysis was performed 3 days later to determine the percentage of GFP+ cells. Viral titer (cfu/ml) was extrapolated from the test point in which non-saturating transduction conditions prevailed (i.e. transduction efficiency 20-80%). Titer (cfu/ml) was calculated as [(% GFP+cells) X (cell number at initial viral exposure)/(viral volume in ml applied)].

### EXAMPLE V

### Transduction of HeLa Cells and Analysis

HeLa cells were plated at 4×10⁴ cells per well in a 6 well dish and allowed to adhere overnight. The cells were transduced with thawed retrovirus (vLTRGFP or vSINGRE5) at an MOI of 15 in whole media (DMEM, 10% heat-inactivated FBS supplemented with 50 units/ml of Pen-Strep). This procedure was repeated daily for three consecutive days. Stably transduced cells were subsequently expanded. No clonal selection was performed, and mixed populations of transduced cells were used for all subsequent experiments. Flow cytometric analysis was performed within two weeks following transduction to ascertain retrovector expression and gene transfer efficiency as measured by GFP fluorescence. In brief, adherent transduced cells were trypsinized and resuspended in RPMI at ˜10⁵ cells per ml. Analysis was performed on a Epics XL/MCL Coulter analyzer. Live cells were gated based on FSC/SSC profile and analyzed for GFP fluorescence.

Glucocorticoid induction assays were conducted using various dilutions of dexamethasone (SABEX Inc., Boucherville, PQ) either in whole media or in corticosteroid depleted FBS-containing media that was generated by charcoal-stripping. In brief, charcoal stripping was performed as follows; 3 g of activated charcoal (Gibco BRL) and 0.3 g of Dextran T40 (Pharmacia Biotechlab, Uppsala, Sweden) are dissolved in 300 ml PBS and pelleted by spinning at 2000 rpm for 10 mins. 10% FBS is then mixed with the charcoal pellet by inversion and incubation at 37° C. for 15 min, then corticosteroid depleted by incubating at 4° C. for 30 min. This procedure is repeated twice with a new charcoal pellet each time. Stripped FBS is then filtered.

Southern blot analysis was performed on 15 μg of overnight KpnI digested genomic DNA extracted from stably transduced cells as well as untransduced control cells. Blots were hybridized with P³² labeled cDNA probes as depicted in FIG. 2, washed and exposed on photographic film. Northern blot analysis was performed on 15 μg total RNA extracted using TRIZOL reagent (Gibco, BRL) from stably transduced cells as well as untransduced control cells. Blots were hybridized with p³² labeled cDNA probes as depicted in FIG. 6, washed and exposed on photographic film.

### EXAMPLE VI

### Harvest, Culture and Transduction of Rat Bone Marrow Stroma

One male inbred Lewis rat (˜200 g) (Charles River Laboratory, Laprairie Company, PQ) was sacrificed by isofluorane inhalation and the hind legs femurs and tibias isolated. Whole marrow was harvested by flushing these bones with DMEM supplemented with 10% FBS and 1% Pen/Strep, and placed in three 150 cm² tissue culture flasks. Following 7 days incubation at 37° C. with 5% CO₂, the non-adherent hematopoietic cells were discarded and the adherent bone marrow stromal cells allowed to expand for an additional 14 days. The rat stromal cells were then plated at a density of 2.5×10⁴ cells per well of a 6-well plate. The next day, media was removed from each well and replaced with 1 ml of media containing 3×10⁵ cfu of thawed vSINGRE5 retrovirus (final MOI of 12) and 6 μg/ml lipofectamine (Gibco, BRL). Stromal cells were also transduced with vLTRGFP retroviral particles at a final MOI of 50 for two consecutive days in the presence of 6 μg/ml polybrene. The resulting mixed population of transduced stroma was subsequently expanded for 4 weeks and a fraction of gene-modified cells was then exposed for 6 consecutive days to dexamethasone at a final concentration of 250 ηM. Flow cytometric analysis to assess GFP fluorescence was performed as described above.

### EXAMPLE VII

### Fluorescence Microscopy

Cells were plated over 22 mm square microscope cover glasses previously placed in wells of 6-well flat bottom tissue culture plates. Once cells reached subconfluency, they were washed with phosphate buffered saline (PBS) three times, fixed by exposing to 3% paraformaldehyde for 15 mins at room temperature, and washed again several times with PBS. The cover glasses were then removed and mounted on precleaned frosted end microscope slides (Fisher Scientific) using gelvatol. Photographs of cells under fluorescence microscopy were taken utilizing a Olympus BX60 microscope attached to a Compaq Deskpro computer. Pro-Series Capture 128 Image-Pro Plus Software was used with an integration time of several seconds for Hela cells and 15 seconds for rat marrow stromal cells. OR with a longer integration time for rat marrow stromal cells versus Hela cells.

### EXAMPLE VIII

### Dexamethasone Regulated Erythropoietin Secretion by Bone Marrow Stromal Cells Following Retroviral Gene Transfer

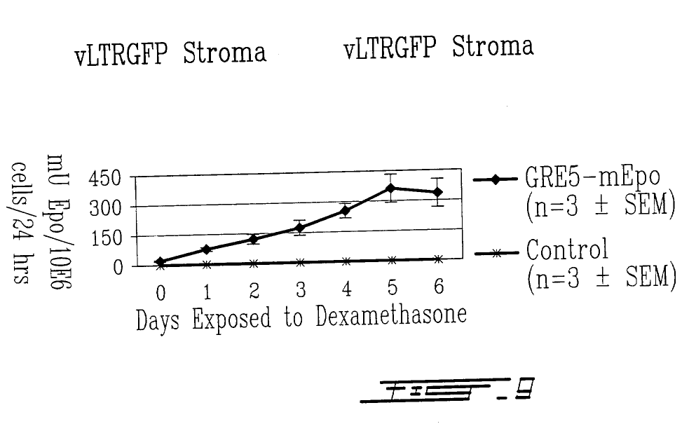

Marrow stromal cells are attractive as a cellular vehicle for the delivery of recombinant proteins, such as erythropoietin (Epo), as they can easily be isolated from bone marrow aspirates, expanded in vitro, transduced with viral vectors, and maintained in vivo. Regulatable expression is vital in therapeutic applications where continuous transgene expression would be deleterious. We have recently demonstrated that marrow stroma can be efficiently engineered with a glucocorticoid-inducible retroviral vector developed in our laboratory and that transgene expression is inducible with dexamethasone and repetitively reversible (Jaalouk et al., Human Gene Therapy 11:1837-1849, 2000). The objective of the present experiment was to explore this drug-inducible genetic switch to provide “on-demand” secretion of Epo. We generated a retroviral construct, GRE5mEpoGFP, comprising the mouse Epo cDNA, an internal ribosome entry site, and the green fluorescent protein (GFP) gene, all under the control of an inducible promoter containing 5 glucocorticoid response elements (GRE5) driving transgene expression in transduced cells. This recombinant plasmid DNA was stably transfected into GP+E86 packaging cells and virus-producers were generated. Bone marrow was harvested from the hind leg femurs and tibias of C57BI/6 mice and 5 days later stromal cells were exposed twice per day for 3 consecutive days for each of 2 weeks to retroparticles. At over 72 hrs post-transduction, cells were exposed to 250 nM dexamethasone for 6 successive days. Throughout this interval, media was collected daily from engineered stroma and evaluated by enzyme linked immunosorbent assay (ELISA) for the amount of secreted Epo. Results are presented in FIG. 9. GRE5-mEpo-GFP transduced stromal cells were noted to secrete increasing levels of Epo attaining 338±69 mU per 10⁶ cells per 24 hrs (mean±SEM, n=3) following 6 day drug exposure. In the absence of dexamethasone only very low level transcriptional activity, hence little “leakiness”, was observed, precisely 20±2 mU Epo/IO⁶ cells/24 hrs. A parallel group of stromal cells was engineered with a control retrovector and likewise exposed to dexamethasone. Epo secretion by these cells remained at normal basal levels, 7±5 mU/10⁶ cells/24 hrs (n=3) throughout the 6 days. These data forecast that GRE5-mEpo modified stroma may serve as a cellular vehicle for dexamethasone regulated production of therapeutic levels of erythropoietin in vivo.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A drug inducible express vector for transfection or administration to an eukaryotic host cell, said vector comprising a therapeutic transgene operably linked to a reporter and to an inducible promoter capable of responding to a transcriptional activator of said host cell when said host cell is exposed to an effective amount of a clinically acceptable drug.

2. An expression vector according to claim 1, wherein said vector consists of a viral vector.

3. An expression vector according to claim 2, wherein said vector consists of a C-type retrovirus or a lentivirus.

4. An expression vector according to claim 3, wherein said vector is capable of integrating into a genome of said host cell.

5. An expression vector according to claim 4, wherein said transcriptional activator consists of a glucocorticoid receptor (GR), and wherein said inducible promoter comprises a glucocorticoid response element (GRE).

6. An expression vector according to claim 5, wherein said inducible promoter consists of a hybrid promoter with five tandem repeats of said GRE and a green fluorescent protein (GFP) reporter.

7. An expression vector according to claim 6, wherein said transgene encodes a cytokine, a hormone, a growth factor, a clothing factor or a chimeric protein.

8. An expression vector according to claim 7, wherein said drug consists of a steroid drug or an analog thereof.

9. An expression vector according to claim 8, wherein said steroid drug consists of dexamethasone.

10. A transplantable eukaryotic host cell for delivering a transgene comprising the expression vector of claim 1 to a patient, said eukaryotic host cell being derived from said patient and capable of engrafting in said patient without immune rejection by said patient and without requiring toxic conditioning.

11. A transplantable eukaryotic host cell according to claim 10, said eukaryotic host cell consisting of a primary cell.

12. A transplantable eukaryotic host cell according to claim 11, wherein said primary cell is selected from the group consisting of a bone marrow stromal cell, a skin fibroblast, a myoblast and an endothelial cell.

13. A transplantable eukaryotic host cell according to claim 12, said primary cell consisting of a bone marrow stromal cell.

14. A system for delivering a transgene to a patient, said system comprising a transplantable eukaryotic host cell according to claim 10 transduced with said expression vector.

15. A method for introducing an expression vector according to claim 1 into a transplantable eukaryotic host cell according to claim 10, comprising infecting said host cell with said expression vector.

16. A method for producing a system according to claim 14, comprising introducing said expression vector into an eukaryotic host cell.

17. A method for regulating expression of a therapeutic transgene product to a patient in need of said therapeutic transgene product, comprising:
a) introducing into a patient a system comprising a transplantable eukaryotic host cell derived from the patient, capable of engrafting in the patient without immune rejection by said patient and without requiring toxic conditioning and transformed with a drug inducible expression vector comprising a therapeutical transgene operably linked to an inducible promoter capable of responding to a transcriptional activator of said eukaryotic host cell when exposed to an effective amount of said drug; and
b) contacting said effective amount of said drug with said eukaryotic host cell, said drug binding to said transcriptional activator of said eukaryotic host cell, thereby inducing said inducible promoter and activating expression of said therapeutic transgene, whereby said expression is regulated.

18. A method according to claim 17, wherein the patient has a mesenchymal disorder.

19. A method according to claim 18, wherein said patient received chemotherapy or radiotherapy prior to step b).

20. A method according to claim 18, wherein the drug consists of a steroid drug or an analog thereof.

21. A method according to claim 19, wherein the steroid drug is dexamethasone.

22. A method according to claim 20, wherein the system is introduced in marrow, spleen, lung, liver or brain of the patient.
